(19) United States
(12) Patent Application Publication
(10) Pub. No.: US 2006/0009825 A1
(43) Pub. Date: Jan. 12, 2006

(54) Method and device for physiotherapy

(76) Inventors: Viatcheslav Mikhailovich Chiriaev, Moscow (RU); Viktor Stepanovich Meleshkov, Moscow (RU); Andrev Aleksandrovich Nazarov, Balashikhinski raion (RU); Vladimir Vasilevich, Lytkarino (RU); Nikolai Vladimirovich Kovalev, Moscow (RU)

Correspondence Address: DELLETT & WALTERS, P. O. BOX 82788, PORTLAND, OR, 97282-0788, US

(21) Appl. No.: 10/507,037

(22) PCT Filed: Oct. 22, 2002
(86) PCT No.: PCT/RU02/00460

§ 371 Date: Jun. 13, 2005

(30) Foreign Application Priority Data
Oct. 29, 2001 (RU) 2001128892

(51) Int. Cl. A61F 2/00; A61N 1/00

(52) U.S. Cl. 607/101; 607/154

(57) ABSTRACT

This invention relates to biology, veterinary and medicine, and more particularly to methods and devices for physiotherapy with the use of electromagnetic radiation in the ultra-high frequency band. The method consists in that patients are irradiated with electromagnetic microwave field at, at least, one frequency belonging to, at least, one of the 18 frequency ranges. The intensity of radiation at the place of location of the patient does not exceed 3.6 μW/cm². The device for carrying out the method comprises, at least, one oscillator ensuring the production of electromagnetic radiation in the 18 frequency ranges, both at the main frequencies and at multiple ones. In the result the effect of pain relief is achieved, inflammatory processes are lessened, processes of regeneration of damaged tissues are stimulated, an affected immune status is corrected, the processes of cell proliferation in neoplasms are stabilized, the lysis of necrotic tissue cells occurs.

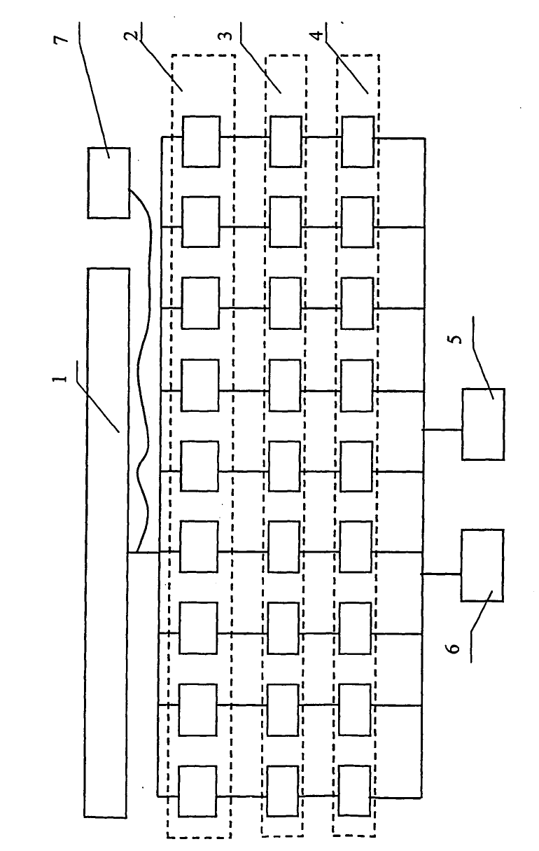

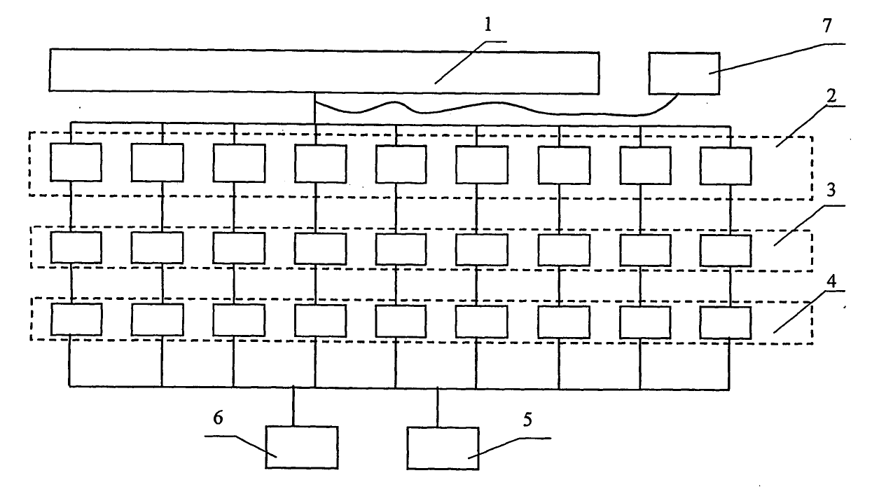

### FIELD OF THE INVENTION

This invention relates to biology, veterinary and medicine, and more particularly to methods and devices for physiotherapy with the use of an oscillatory component of electromagnetic radiation in the ultra-high frequency band. The biophysical action of the invention is realized at the level of biological subunits and is intended to ensure the formation of immunomodulatory, anti-inflammatory, lytic, analgesic and sedative effects, as well as the effect of stabilizing cell proliferation in neoplasms. Therefore, the invention may be used for physiotherapy purposes in various fields of medicine of both non-oncological and oncological profiles, e.g., in oncosurgery, oncourology, gynecology, as well as in combined methods of radio beam therapy of malignant neoplasms and in other fields of medicine. Moreover, the invention may be used for treatment of diseases associated with psychoneurological disorders, vegetative disorders of the cardio-vascular system, and diseases associated with disorders in the functions of the immune system and the endocrine system.

### PRIOR ART

At present great attention is paid to studying the effect of low-intensity electromagnetic radiation (up to 10 μW/cm²) on biological objects, since it has been reliably proven that microwave frequencies (UHF) act on biological molecules, on submolecular systems (polypeptides, amino acids, proteins), on flow property of blood, on biochemical and physiological activity indices of organs and systems of living organisms, on behavior, education processes and memory.

Among the identified regularities in the action of low-intensity microwave fields it may be mentioned that low-intensity electromagnetic fields of radio-frequency region (LIEFRFR) are capable of acting on the processes of biochemical reactions of the intracellular metabolism, on the activity of protein enzymes, on the neurohumoral regulation of the vegetative nervous system. Electromagnetic fields change the dielectric properties of a medium and, thus, may change the speed of electric and wave information propagation in biological systems and act on the reproductive abilities of the organism, and if their action is prolonged they can act on the processes of the DNA reduplication and act, directly or indirectly, on the processes of transfer of genetic information. Also, if the LIEFRFR action is prolonged, transformations in polypeptide chains of biological subunits are possible up to breakes in such chains and formation of various peptidases and kinins. The latter are capable of influencing the biochemical orientation and speed of various metabolic processes ensuring the maintenance of the hormonal and immune balances, the cell apoptosis and phagocytosis, the necrotic tissue lysis, removal of inflammatory responses, acceleration of processes of damaged tissue healing, the regulation of the vegetative nervous system and the maintenance of many other vital physiological processes of the biological system survival.

But the study results and their interpretation, due to their certain fragmentary and, often, contradictory nature, do not enable by now to create an integral concept on the perception of low electromagnetic microwave radiation and the nature of the frequency-dependent character of this process. There is no single view on the molecular-cell mechanisms of the electromagnetic radiation influence on biological objects. Thus, in the classical physiotherapy, in order to justify the therapeutic exposure to electromagnetic radiation, the theory of resonance is used, according to which the energy of microwave radiation is absorbed in a case where the radiation frequency coincides with the characteristic frequencies of the relaxation of bound water dipole molecules as well as protein and glycolipid side groups of the plasmalemma.

Finally, in a case of the exposure oscillatory component, activation of the cell breathing and the enzymatic activity, conformation restructuring of the plasmalemma glycolipids, a change in permeability and the functional properties of membranes in the irradiated tissues occur. Apart from the idea on the energy character of interaction between an electromagnetic field and microstructures, where the main role is allotted to the formation of a response, a possibility of information interaction between an electromagnetic field and elements of the biological system is considered. From this point, of special interest is the process of influencing various organs and systems with low-intensity microwave fields modulated in the frequency range of one's own biological rhythms. It is supposed that the effect of electromagnetic fields, which are modulated by a frequency of the irradiated organ's own biorhythms, may enhance the biological effect of the electromagnetic field. There also exists a possibility of creating a low, highly structured electromagnetic field carrying adequate information biologically significant for certain structures. Thus, for example, a low pulse-modulated signal, which frequency characteristics are set by the parameters of the electric activity of the lymphoid tissue (spleen), induces the formation of low molecular, endogenous immunostimulants comprising peptide bonds and amino acids.

Known in the art is a method of forming stimulating action, under which a human being exposed, in particular, to microwave radiation, which is modulated by a low-frequency signal having the frequency spectrum 1/f, where f is a spectral frequency (RU 2053803, C1, A 61 N 5/00, 1996). The said method is intended for physiotherapy and rehabilitation of human beings. The implementation of the said method presupposes the availability of an additional source for signal modulation in the device comprising the signal source. The parameters of a signal of a selected type, e.g. microwave radiation, are transformed into a signal of random pulse sequence, which ensures the harmonic resonance of the exposure with the human being biorhythms due to complete correlation of the exposure in the spectrum with the biological rhythms of processes ongoing in the human organism. It leads to increasing the therapeutic effect as well as to accelerating the rehabilitation of a human being. But the said method lacks a link between the formed electromagnetic pulses and the inner electromagnetic processes ongoing in the patient's organism, and, as a consequence, long-term therapy at some types of diseases.

Also known in the art is a method of physiotherapy consisting in exposing a human being to microwave radiation in the region from 300 MHz to 300 GHz, which is modulated with the frequency corresponding to the frequency of non-electric heart signals (RU 2051703, C1, A 61 N 5/00, 1996). The said method is carried out with the use of a device comprising an electromagnetic oscillator, a modulator, a device for applying electromagnetic oscillations to a patient, transducers of electric and non-electric signals. The presence of these assemblies enables to modulate the intensity of high-frequency electromagnetic oscillations, as applied to a patient, synchronously and in phase with the electric processes in the organism of the patient. The organization of this correlation enables to use the energy of an electromagnetic field more purposefully and efficiently when acting on tissues and organs, and, consequently, shortens the period of therapy.

The closest to the described method, as to the technical essence and the achieved result, is the method of physiotherapy consisting in that a patient is irradiated by electromagnetic microwave radiation (RU 2134597, C1, A 61 N 5/00, 1999). The device for physiotherapy comprises an oscillator producing electromagnetic microwave radiation, which is connected to an irradiating antenna device. The device also comprises a digital noise generator connected to the input of a low-pass filter. The digital generator produces a random sequence of pulses for forming low-intensity microwave radiation having the frequency parameters coinciding with the parameters of natural movements of organism cells. The said sequence is fed to the said low-pass filter where it is transformed into an analogue signal of arbitrary form and, after its amplification, is fed to the said microwave oscillator. Therefore, the output capacity of the microwave oscillator varies according to a pseudorandom process and coincides with the frequency of natural micro-movements of the organism cells, i.e., when the field acts on the organism spontaneous micro-movements of organs and tissues are activated, which, in its turn, improves and accelerates the therapeutic effect.

Thus, modulation of microwave radiation by various methods increases the therapeutic effect. But, in modulating microwave radiation during the irradiation the efficiency of microwave radiation action on tissues of biological objects varies periodically. It is conditioned by the fact that the receptiveness of various tissues is changed depending on the frequency of microwave radiation.

### DESCRIPTION OF THE INVENTION

The objective of this invention is to develop and create a method and a device that will enable to ensure physiotherapy action on organisms of animals and human beings. In the result of solving this task it is supposed to form such physiotherapy effects, through which the effect of pain relief is achieved, inflammatory processes are lessened, processes of regeneration of damaged tissues are stimulated, an affected immune status is corrected, the processes of cell proliferation in neoplasms are stabilized, the lysis of necrotic tissue cells occurs, prophylaxis of complication resulted from X-ray therapy and chemotherapy in cases of oncological diseases is ensured, tolerance to the influence of various damaging factors on organisms of animals and human beings is increased.

The said results are achieved owing to that the method of physiotherapy consists in that the patient is irradiated with electromagnetic microwave radiation at, at least, one frequency belonging to, at least, one of the frequency ranges selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz, and the exposure to radiation is carried out with the modulation frequency from 4 Hz to 200 Hz, and the intensity of electromagnetic radiation produced by the oscillators through the irradiating antenna device at the place of location of the patient is set at a value not more than 3.6 μW/cm².

For this purpose in the physiotherapy device comprising the oscillator for producing electromagnetic microwave radiation, which is connected to the irradiating antenna device, the condition is fulfilled for ensuring the possibility of frequency-modulating the carrier frequency in the range from 4 Hz to 200 Hz and functioning in the frequency range selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz, the density of the electromagnetic radiation power flow produced by the oscillator through the irradiating antenna device at the place of location of the patient being not more than 3.6 μW/cm².

A distinctive feature of this invention is the possibility of acting on a biological system with at least one frequency spectrum of low-intensity electromagnetic radiation, which is formed either by one or by several oscillators, but with the same frequency of signal modulation and through one antenna device. This result is achieved owing to the use of one or several oscillators capable of forming both the frequency spectrum to which the oscillator is directly set and harmonic oscillations multiple to the main resonant frequencies. Moreover, it has been experimentally identified that the therapeutic effect can be noticed when irradiating a patient with electromagnetic microwave radiation at any frequency relating to one of the frequency ranges selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz. Any combination of frequencies relating to the said frequency ranges increases the effect of exposure.

Furthermore, the device may comprise at least one additional oscillator of electromagnetic microwave radiation, which is connected to an irradiating antenna device and functions in a frequency range selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz. And the radiation thus formed may be frequency-modulated to one of the frequencies selected from the range from 4 Hz to 200 Hz.

The obtaining of a qualitatively new level of exposure to electromagnetic microwave radiation is ensured by the possibility of combining several frequency spectra with different ranges at one modulation frequency of the formed signal and through one antenna device. It has been experimentally determined that a significant condition is the necessity of modulating the selected frequencies within the limits from 4 Hz to 200 Hz. An important feature of physiotherapy procedures is the threshold level of intensity of acting with electromagnetic radiation oh biological objects.

If the intensity of electromagnetic microwave radiation at the place of location of an object exceeds 3.6 μW/cm², the supersaturating effect begins forming. In the result the physical properties (dielectric permittivity, electrical conduction) of tissues, in which the electromagnetic wave propagates, are changed. This results in changing the nature of the described action on a biological object, which, later on, significantly lowers the quality of the achieved effects.

Furthermore, a patient is irradiated with electromagnetic microwave radiation once daily or once every two days for 0.5-3 hours, the total irradiation time being from 8 to 24 hours. It is advisable to provide the physiotherapy device with a portable irradiating antenna device. The biophysical aspect of forming a therapeutic effect from this type of exposure consists, seemingly, in that the identified narrow variation ranges of electromagnetic radiation correspond to resonance frequencies of individual radicals in the biologically significant macromolecules. The macromolecules, which have become the mediated “targets” for low-intensity electromagnetic radiation relate to the elements of intracellular signals bearing relation to taking decisions vital for the organism by the cell.

The above advantages as well as the specific features of this invention are explained by the description of its embodiment with references to the appended drawing.

### BRIEF DESCRIPTION OF THE DRAWING

The drawing shows the general layout of the physiotherapy device.

### DESCRIPTION OF THE PREFERRED EMBODIMENT

The described method is implemented through the physiotherapy device comprising the common irradiating antenna device 1. To the device 1 the oscillators 2 are connected, the number of which is at least 1 and not more than 18, which is conditioned by the number of frequency ranges for irradiating a patient. The oscillators are made with the possibility of producing both a resonant frequency and its harmonic. This technical solution, with due regard to frequency deviation, enables to use a lesser number of oscillators for irradiating a patient in all the 18 frequency ranges.

Each oscillator 2 is connected to the frequency modulation unit 3, which outputs are connected to the individual control units 4. The outputs of the control units 4 are connected to the common control panel 5 and to the program control unit 6. The common irradiating antenna device 1 may be installed permanently at a distance from 1.5 to 8 meters from the place of location of a patient. If it is necessary to irradiate a local area on a patient, the device is provided with the portable irradiating antenna device 7.

Each oscillator 2 is preset with the use of the individual control unit 4 for radiation with a spectrum of oscillations at fixed frequency ranges.

The individual control unit 4 enables to set the modulation frequency for the oscillators 2. The generator output is selected in advance by way of simple experiments so that at the place of location of a patient the output of all the operating oscillators does not exceed 3.6 μW/cm². The selection of a combination of the operating oscillators, the exposure time, the modulation frequency and the control over the operation of the oscillators may be done manually with the use of the individual control units 4 and the common control unit 5, or, in the automatic mode, with the use of program control unit 6.

Examples of physiotherapy of patients, which have been obtained in the course of clinical studies, illustrate the use of the described physiotherapy method and device.

### EXAMPLE 1

Patient T., aged 54, the diagnosis - chronic prostatitis, had this disease for 5 years, the previous treatments gave only temporary effect. The therapy course using electromagnetic microwave radiation was carried out for 3 hours every two days for 15 days (8 sessions in total). Each session was carried out with simultaneous operation of all the oscillators at the following frequencies: 1,550 MHz, 1,251 MHz, 1,101 MHz, 977 MHz, 720 MHz, 591 MHz, 554 MHz, 473 MHz, 465 MHz, 417 MHz, 306 MHz, 282 MHz, 219 MHz, 117 MHz, 90 MHz, 45 MHz. The modulation frequency was 4 Hz. The total power intensity of the operating oscillators was 3.6 μW/cm².

Subjectively and objectively—the effect was positive; the pains were removed, the urination did not have specific features, the nycturia disappeared. This effect was observed for more than half a year from the time of completing the course.

### EXAMPLE 2

Patient S., aged 52. The diagnosis “cancer of esophagus”, the 4th stage, at the arrival. After the operation the inconsistency of the sutures of the relieving gastrostoma (persistent wound) was noticed. The additional treatment—radiotherapy. The patient received a course of electromagnetic radiation with the following times of exposure: the 1st day—1 hour, the following 6 days—2 hours each day. The exposure was carried out at the following frequencies: 1,610 MHz, 1,295 MHz, 1,126 MHz, 940 MHz, 718 MHz, 592 MHz, 547 MHz, 470 MHz, 445 MHz, 435 MHz, 301 MHz, 280 MHz, 213 MHz, 117 MHz, 93 MHz, 43 MHz at the modulation frequency 72 Hz. The total power intensity of the operating oscillators was 3.0 μW/cm². After the treatment course the patient's condition improved, the physical activity increased. No acute conditions due to radiation appeared, acceleration of reparative processes was observed.

### EXAMPLE 3

Patient K., aged 52. The diagnosis—rheumatoid arthritis. A treatment course with the use of the invention was administered. The treatment course consisted of six 2-hour sessions carried out every two days. Each session was carried out with the oscillators operated jointly at the following frequencies: 1,110 MHz, 961 MHz, 559 MHz, 420 MHz, 273 MHz, 215 MHz, 121 MHz at the modulation frequency 155 Hz. The total power intensity of the operating oscillators was 3.6 μW/cm². The applied therapy enabled to lower the painful sensations, to increase the degree of freedom when bearing physical loads, and prevent seasonal acute conditions of the disease.

### EXAMPLE 4

Patient T.(woman), aged 48, the disease—chronic adnexitis with acute conditions accompanied by pains in the lower part of the stomach, temperature increases, which required anti-inflammatory therapy. A physiotherapy course with the use of low-intensity electromagnetic microwave radiation was administered and carried out in February 1999: for 3 hours daily for 15 days. Each session has been carried out with simultaneous operation of all the oscillators at the following frequencies: 1,140 MHz, 940 MHz, 540 MHz, 445 MHz, 420 MHz, 309 MHz at the modulation frequency 200 Hz. The total power intensity of the operating oscillators has been 2.5 μW/cm². Against the background of the standard anti-inflammatory therapy no acute conditions of the disease were observed during next 9 months.

### EXAMPLE 5

Patient K. (woman), aged 57, got domestic chemical burn on her lower leg. The patient received a conservative therapy with the use of salve dressings and therapy sessions with low-intensity electromagnetic microwave radiation for 2 hours daily for 5 days. The sessions were carried out with the simultaneous operation of the oscillators at the frequency 540 MHz at the modulation frequency 125 Hz. The total power intensity of the operating oscillators has been 1.8 μW/cm². The burn was liquidated. After the operation the radiation course was repeated at the frequency 90 MHz for 2 hours daily every two days for two weeks.

### EXAMPLE 6

Patient V. (woman), aged 51, the diagnosis—cancer of the piriforrn sinus, 4th stage, the condition after radiation therapy, a residual tumor. She was operated. The postoperative period: inflammatory process in the area of the implantation grafting, abscess, persistent wound, pains of the 2nd degree. A therapy course with low-intensity electromagnetic radiation was carried out for 4 days: 1st day—50 minutes, the next 3 days—2 hours daily. The exposure was carried out with the simultaneous operation of the apparatus at the following frequencies: 1st day—1,104 MHz, 540 MHz, 285 MHz, 211 MHz, 117 MHz at the modulation frequency 45 Hz; 2nd, 3rd and 4th days—1,130 MHz, 965 MHz, 559 MHz, 430 MHz, 420 MHz, 301 MHz, 273 MHz, 219 MHz, 113 MHz at the modulation frequency 170 Hz. The total power intensity of the operating oscillators has been 3.6 W/cm². After the 4-day course of exposure the physical mobility increased, the abscess in the wound decreased (i.e., correction of the affected immune status occurred), the pain syndrome was lowered to the 1st degree.

### EXAMPLE 7

Patient A. (woman), born in 1985, a metastasis in the chiasmatic region. A brain tumor in the occipital region, the third recurrence. Cachexia. The immunomodulatory chemotherapy was administered to the patient, but it did not give the expected results. After carrying out a course of therapy by low-intensity electromagnetic radiation for 4 days at the frequencies 213 MHz, 425 MHz, 960 MHz, which were switched on successively and acted on the patient I hour each, the cachectic symptoms (nausea, vomiting) were removed, and the patient's resistance to physical loads was increased. The total power intensity of the operating oscillators has been 3.2 W/cm². When analyzing the immunograms a statistically reliable positive dynamics was identified, which evidenced the normalization of the immunological status.

### EXAMPLE 8

Patient Ya, born in 1985, with the diagnosis “a glioblastoma in the right temporal lobe with the parastem spread, the total spinal marrow affection, flail legs”. The radiation chemotherapy was planned for the patient, which could hardly be carried out due to negative indices in the patient's immunological status. In the result of using low-intensity electromagnetic radiation in combination with the radiation chemotherapy appearance of undesirable complications (pronounced leucopenia) was avoided and a full course of radiation therapy was carried out. Moreover, a positive neurological dynamics was noticed in the form of partial normalization of the functioning of the pelvic bodies, increase in the movement volume and an improvement of the tactile sensation. The low-intensity electromagnetic radiation treatment was carried out for 6 days at the following frequencies: 1,158 MHz, 1,270 MHz, 1,126 MHz, 960 MHz, 720 MHz, 576 MHz, 547 MHz, 480 MHz, 454 MHz, 425 MHz, 309 MHz, 280 MHz, 213 MHz, 146 MHz, 117 MHz, 90 MHz, 45 MHz, which were switched on successively with the 10-minute exposure at each frequency. The total power intensity of the operating oscillators has been 3.6 μW/cm².

### EXAMPLE 9

Patient A. (woman), born in 1939, the diagnosis—stomach cancer, metastases in bones of the skeleton. The indication for the low-intensity electromagnetic radiation therapy was the chronic pain syndrome stopped by tromal (CPS of the 2nd degree). The treatment with low-intensity electromagnetic radiation was carried out for 6 days at the frequencies 1,155 MHz, 1,273 MHz, 1,124 MHz, which were switched on successively, with the 60-minute exposure for each frequency. The total power intensity of the operating oscillators has been 2.2 μW/cm². After the treatment the pains were stopped, and the patient was saved from the necessity of buying and taking any analgesics for a long period of time (3 months). When the pains resumed (3 months later), the patient underwent the repeated course of low-intensity electromagnetic radiation therapy, but that time already in combination with frequencies of a longer-wave spectrum: 45 MHz, 90 MHz, 117 MHz, 213 MHz, 280 MHz. The total power intensity of the operating oscillators has been 3.6 μW/cm². The frequencies were combined in an arbitrary manner for each next treatment session. In the result of the repeated treatment not only the pain syndrome was removed, but also the physical activity improved, and the patient gave up the auxiliary means for movement (a crutch).

### EXAMPLE 10

Patient Zh. (woman). The diagnosis—carcinoma of the right mammary gland with metastases in bones of the skeleton. The indication for the low-intensity electromagnetic radiation therapy was the chronic pain syndrome of the 2nd degree. After the treatment with low-intensity electromagnetic radiation at the frequencies 1,126 MHz, 547 MHz, 425 MHz, 117 MHz, 45 MHz, which were switched on successively after switching off the previous frequency and acted with the 35-minute exposure each, the pain syndrome lessened and the degree of movement freedom increased. The total power intensity of the operating oscillators has been 2.8 μW/cm².

In the course of the clinical trials the efficiency of the physiotherapy with low-intensity electromagnetic radiation at the above frequencies was found to be 62%.

### INDUSTRIAL APPLICABILITY

This invention is industrially applicable and may be used most successfully in biology, veterinary and medicine, namely for physiotherapy with the use of the oscillatory component of electromagnetic radiation in the ultra-high frequency band. The biophysical action of the invention is realized at the level of biological subunits and is intended to ensure the formation of immunomodulatory, anti-inflammatory, lytic, analgesic and sedative effects, as well as the effect of stabilizing cell proliferation in neoplasms. Therefore, the invention may be used for physiotherapy purposes in various fields of medicine of both non-oncological and on-cological profiles and does not require creation of special unknown devices.

1. A physiotherapy method consisting in that a patient is irradiated with electromagnetic microwave radiation, characterized in that the said patient is irradiated with electromagnetic microwave radiation at, at least, one frequency belonging to, at least, one of the frequency ranges selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz, and the exposure to radiation is carried out with the modulation frequency from 4 Hz to 200 Hz, and the intensity of electromagnetic radiation produced by the oscillators through the irradiating antenna device at the place of location of the patient is set at a value not more than 3.6 μW/cm².

2. The physiotherapy method according to claim 1, characterized in that in one session the patient is irradiated with electromagnetic microwave radiation for the period from 0.5 to 3 hours.

3. A physiotherapy method according to claim 1, characterized in that the patient is irradiated with electromagnetic microwave radiation daily or every other day with the total irradiation time from 8 to 24 hours.

4. A physiotherapy device comprising an oscillator for producing electromagnetic microwave radiation, which is connected to an irradiating antenna device, characterized in that the said oscillator for producing electromagnetic microwave radiation, which is connected with the said antenna device, is made with the possibility of modulating the frequency in the range from 4 Hz to 200 Hz and operates in the frequency range selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz, and the density of the electromagnetic radiation power flow produced by the oscillator through the irradiating antenna device at the place of location of the patient being not more than 3.6 μW/cm².

5. A physiotherapy device according to claim 4, characterized in that it comprises at least one additional oscillator producing electromagnetic microwave radiation, which is connected with the antenna device, is made with the possibility of modulating the frequency in the range from 4 Hz to 200 Hz and operates in the frequency range selected from the following series: from 1,554 MHz to 1,618 MHz, from 1,245 MHz to 1,295 MHz, from 1,104 MHz to 1,150 MHz, from 940 MHz to 978 MHz, from 706 MHz to 736 MHz, from 568 MHz to 592 MHz, from 528 MHz to 563 MHz, from 470 MHz to 492 MHz, from 445 MHz to 465 MHz, from 415 MHz to 435 MHz, from 301 MHz to 317 MHz, from 274 MHz to 291 MHz, from 209 MHz to 219 MHz, from 140 MHz to 152 MHz, from 113 MHz to 121 MHz, from 85 MHz to 95 MHz, from 39 MHz to 51 MHz, from 15 MHz to 21 MHz.

6. A physiotherapy device according to claim 4, characterized in that the said irradiating antenna device is installed at a distance from 1.5 to 8 meters from the place of location of the patient.

7. A physiotherapy device according to claim 4, characterized in that the said device is provided with a portable irradiating antenna device.

8. A physiotherapy method according to claim 2, characterized in that the patient is irradiated with electromagnetic microwave radiation daily or every other day with the total irradiation time from 8 to 24 hours.

9. A physiotherapy device according to claim 5, characterized in that the said irradiating antenna device is installed at a distance from 1.5 to 8 meters from the place of location of the patient.

10. A physiotherapy device according to claim 9, characterized in that the said device is provided with a portable irradiating antenna device.

11. A physiotherapy device according to claim 5, characterized in that the said device is provided with a portable irradiating antenna device.

12. A physiotherapy device according to claim 6, characterized in that the said device is provided with a portable irradiating antenna device.
